(19) United States
(12) Patent Application Publication
(10) Pub. No.: US 2004/0039391 A1
(43) Pub. Date: Feb. 26, 2004

(54) Bone treatment employing reduced pressure

(76) Inventors: Louis C. Argenta, Winston-Salem, NC (US); Michael J. Morykwas, Winston-Salem, NC (US); Lawrence X. Webb, Winston-Salem, NC (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/227,161

(22) Filed: Aug. 23, 2002

(51) Int. Cl. A61F 5/00

(52) U.S. Cl. 606/86

(57) ABSTRACT

A method of treating bone tissue damage is provided which includes applying a reduced pressure to a damaged bone tissue sufficient in time and magnitude to promote healing of the damaged bone tissue. A bone tissue treatment apparatus is provided in which a fluid impermeable tissue cover is sealed over a bone tissue site. A screen in the form of an open-cell foam screen or a rigid porous screen is placed beneath the tissue cover over the damaged bone tissue. A vacuum pump supplies suction within the tissue cover over the treatment site.

### FIELD OF THE INVENTION

The present invention relates to an apparatus and method for treating bone tissue by applying reduced pressure to the bone tissue.

### BACKGROUND OF THE INVENTION

Promoting the growth of bone tissue, especially bone tissue damaged through trauma or disease, has long been an area of concern in medical practice. Such damage or disease, including complications due to infection, may hinder or prevent healing of an injury due to a lack of bone tissue growth. Certain diseases and injuries involve affected bone tissue that cannot heal spontaneously. Such is the case, for example, for an open pilon fracture of bone tissue. Historically, a pilon fracture involves a high complication rate. Such complications include infection, nonunion, failure to obtain or maintain a reduction of the joint surface, and early and late arthritis. Under such conditions, failure to achieve sufficient healing of the pilon fracture could necessitate amputation.

In the 1970s and early 1980s the prescribed treatment for most pilon fracture injuries was open reduction and internal fixation, usually with a metaphysical bone graft. Reports of high complication rates with this approach prompted many surgeons to use indirect methods such as bridging external fixation and to limit the surgery to what was necessary for the joint reduction. Awareness of the issues of timing has prompted some to use a staged procedure, with bridging external fixation initially, followed by open but limited surgery. The incisions are dictated by fracture patterns, and the timing is dictated by resolution of the soft tissue envelope.

However, despite these approaches, cases arise where a major complication, e.g., a deep infection, can develop. Depending on the patient's medical condition, such as the condition of local blood vessels, customary treatment by application of a free muscle flap may be inappropriate. In such instances, traditional treatment offers a poor prognosis for salvage of the affected tissue. In such cases, where there is a likelihood of an infected nonunion and its associated pain, deformity and poor function, amputation is the appropriate and preferred medical treatment. Thus, it could be a great advance to the medical practice to provide an apparatus and method to promote healthy bone tissue growth under such circumstances to avoid the drastic treatment of amputation.

As further example, diseases such as cancer often result in bone tissue damage that does not heal spontaneously, and treatment of such resulting bone tissue damage would benefit from an apparatus and method to promote bone tissue growth. For example, many patients who experience injuries or suffer from bone cancer require replacement of a missing piece of bone. Current techniques for bone replacement include: moving a piece of the bone from an uninjured site to the injured site; use of cadaver bone; or the use of metal rods or plates. These options are not always possible due to the potential for defect from the bone donor site, or the lack of availability of cadaver bone. For reasons such as these, the growth of new healthy bone tissue would provide a significant advance the treatment options in such cases.

### SUMMARY OF THE INVENTION

In accordance with the present invention a bone tissue treatment apparatus is provided for treating bone tissue by applying reduced pressure (i.e. pressure that is below ambient atmospheric pressure) to damaged bone tissue in a controlled manner for a selected time period. Damaged bone tissue may include, for example, bone tissue injured by trauma or by disease. The application of reduced pressure to damaged bone tissue provides such benefits as faster healing or healing that would otherwise not occur in the absence of treatment with reduced pressure. For example, injuries that have exhibited positive response to treatment by the application of reduced pressure include an open pilon variety fracture in which new bone material has been grown by the application of reduced pressure, where such growth would not have been expected without the application of reduced pressure.

A bone tissue treatment apparatus in accordance with the method of the present invention includes a reduced pressure application appliance which is applied to a treatment site at which there is damaged bone tissue. The reduced pressure application appliance includes a fluid impermeable tissue cover for covering and enclosing the damaged bone tissue. The appliance may optionally include sealing means for sealing the tissue cover to the surrounding tissue of the damaged bone tissue in order to maintain reduced pressure in the vicinity of the damaged bone tissue during treatment. When the tissue cover is sealed in position over the damaged bone tissue site, a generally fluid-tight or gas-tight sealed enclosure is formed over the damaged bone tissue site. The sealing means may be in the form of an adhesive applied to the underside of the tissue cover for sealing the tissue cover to tissue proximate the damaged bone tissue. The sealing means may also include a separate sealing member such as an adhesive strip or a sealing ring in the form of a tubular pad or inflatable cuff secured to the tissue cover for positioning around the periphery of the damaged bone tissue. In selected embodiments, the reduced pressure within the sealed enclosure under the tissue cover may itself serve to seal the tissue cover in position at the damaged bone tissue site. The reduced pressure appliance may optionally include a suction port for supplying reduced pressure within the sealed volume enclosed beneath the tissue cover. The suction port may be in the form of a nipple on the tissue cover. Alternatively, the suction port may be in the form of a tube attached to the tissue cover or provided as a feed through beneath the tissue cover. The appliance may also include a porous tissue screen for placement at a location between the tissue cover and the damaged bone tissue. The tissue screen may be sufficiently porous to permit gas flow to the damaged bone tissue. The porous screen may be in the form of an open-cell foam material, including a sponge, for placement in the damaged bone tissue. The porous screen may also include a rigid or semi-rigid screen for overlying the damaged bone tissue.

A vacuum system is connected with the reduced pressure appliance in order to provide suction or reduced pressure to the appliance. For this purpose, the vacuum system includes a suction pump or suction device for connection with the suction port of the appliance for producing the reduced pressure at the damaged bone tissue site. The vacuum system may include a section of hose or tube, such as a vacuum hose, that interconnects the suction device with the suction port of the appliance to provide the reduced pressure at the damaged bone tissue site. The apparatus may also include a control device for controlling the pump and for providing intermittent or cyclic production of reduced pressure.

In a particular embodiment of the invention, the tissue cover for the reduced pressure appliance may be in the form of a gas impermeable covering sheet of flexible polymer material, such as polyethylene, having an adhesive backing that provides the seal, for securing the sheet over the damaged bone tissue site to provide a gas-tight or fluid-tight sealed enclosure over the damaged bone tissue site. The vacuum system of the damaged bone tissue treatment apparatus may include a suction pump having a vacuum hose that is connected with a suction tube serving as a suction port for the appliance. The suction tube for the appliance runs beneath the cover sheet that is sealed in position over the damaged bone tissue site and into the fluid-tight enclosure provided under the cover sheet. An adhesive backing on the cover sheet is used to provide a fluid-tight seal around the feed through for the suction tube at the damaged bone tissue site. Within the enclosure, the suction tube is connected with a piece of open-cell foam for placement proximate the damaged bone tissue. The open-cell foam functions to more uniformly apply reduced pressure or suction over the damaged bone tissue site while holding the cover sheet substantially out of the damaged bone tissue during the application of reduced pressure at the enclosed damaged bone tissue site.

A method of treatment of damaged bone tissue is provided which includes the steps of applying a reduced pressure to a bone defect and maintaining the reduced pressure until new bone tissue has grown at the defect to provide a selected stage of healing. The method can be carried out by securing a reduced pressure appliance to the treatment site and then maintaining a substantially continuous or cyclical reduced pressure within the appliance until the damaged bone tissue has reached a desired improved condition. A selected state of improved condition may include formation of a neo-osteoid tissue. It may be preferable to change the appliance periodically during treatment. The method may desirably be practiced using a reduced pressure from 0.01 so as to produce a resulting absolute pressure from 0.01 to 0.99 atmospheres, and more desirably a resulting absolute pressure ranging between 0.5 to 0.95 atmospheres or a resulting absolute pressure ranging between 0.5 and 0.85 atmospheres.

### BRIEF DESCRIPTION OF THE DRAWINGS

The foregoing summary, as well as the following detailed description of the preferred embodiments of the present invention, will be better understood when read in conjunction with the appended drawings, in which:

FIG. 1 is a schematic elevational view of a bone tissue apparatus in accordance with the present invention in which a reduced pressure appliance shown in partial section, includes a flexible, fluid impermeable tissue cover sealed over damaged bone tissue and a foam screen positioned proximate the damaged bone tissue, and in which a vacuum system provides reduced pressure within the tissue cover of the appliance; and

FIG. 2 is a schematic cross-sectional view of a reduced pressure appliance comprising an open-cell polymer foam screen, a flexible hose for connecting the foam screen with a vacuum system, and an adhesive-backed flexible polymer sheet overlying the foam-hose assembly to provide a seal over damaged bone tissue.

### DETAILED DESCRIPTION OF THE PREFERRED EMBODIMENTS

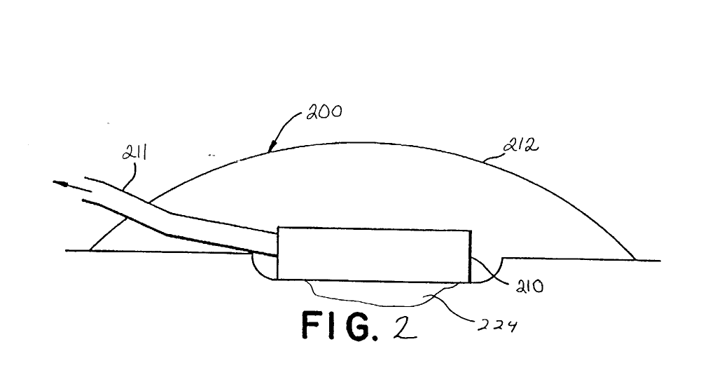

In accordance with the present invention, a bone tissue treatment apparatus is provided for treating damaged bone tissue by application of reduced pressure (i.e., below atmospheric pressure) so that suction may be applied to a damaged bone tissue site in a controlled manner for a selected time period. As schematically shown in FIG. 2, a bone tissue apparatus includes a reduced pressure appliance, generally designated 200, which is applied to a damaged bone tissue site to treat the damaged bone tissue 224 through the application of reduced pressure. The appliance 200 is sealed in position over the damaged bone tissue site to create a generally-fluid-tight or gas-tight enclosure over the damaged bone tissue site.

The appliance 200 may include a substantially flat section of open cell polyester foam section 210 (Fischer Scientific, Pittsburgh, Pa. 15219) sufficiently large to cover a region surrounding the damaged bone tissue 224, a flexible hollow tube 211 (Fischer Scientific) inserted into the open cell foam section 210 and joined thereto with an adhesive and extending to attach at its opposite end with a Gast Vacuum pump (Fischer Scientific), and an Ioban adhesive sheet 212 (Minnesota Mining and Manufacturing, St. Paul, Minn. 55144) overlying the foam section 210 and tubing 211 and adhered to the tissue surrounding the damaged bone tissue, thus forming a seal that allows creation of a vacuum when the suction pump operates. Such an appliance 200 would most preferably be packaged in a sterile condition to ameliorate the need for sterilization of the apparatus prior to use. The adhesive sheet 212 may be packaged separately from the foam-tube assembly 210 and 211. A suitable wound treatment apparatus is also available from Kinetic Concepts International of San Antonio, Tex.

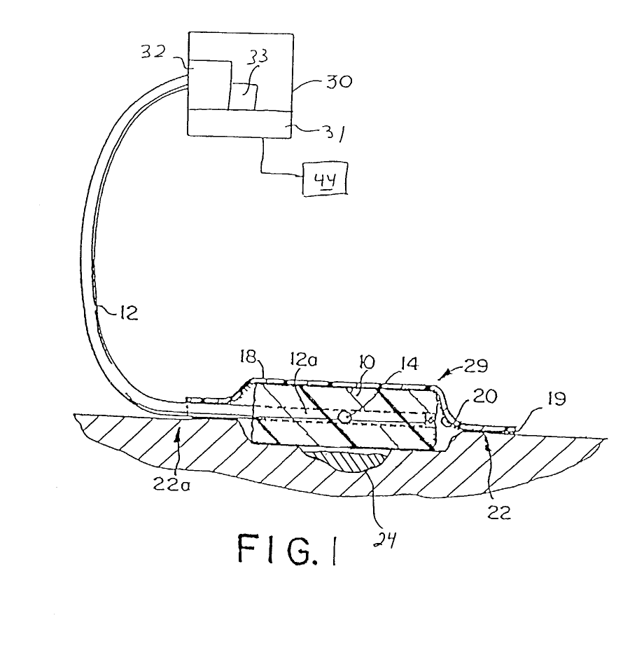

Referring to FIG. 1, a bone tissue treatment apparatus, generally designated 25, is depicted having a reduced pressure appliance 29 for enclosing a damaged bone tissue site to provide a fluid-tight or gas-tight enclosure over the damaged bone tissue site to effect treatment of a damaged bone tissue 24 with reduced pressure. The damaged bone tissue treatment apparatus 25 includes a reduced pressure appliance, generally designated 29, which is applied to and sealed over a damaged bone tissue site in order to enclose the damaged bone tissue site for treatment with suction or reduced pressure within a sealed generally fluid-tight or gas-tight enclosure. For the purpose of creating suction within the appliance 29, the appliance 29 is connected with a vacuum system, generally designed 30, to provide a source of suction or reduced pressure for the sealed appliance 29 at the damaged bone tissue site. The vacuum system 30 may include a suction device 31 and an optional fluid collection device 32 intermediate the hose 12 and suction device 31. The suction device 31 produces a source of reduced pressure or suction which is supplied to the reduced pressure appliance 29 by suction tubing 12. The fluid collection device 32 functions to collect any exudate that may be aspirated from the tissue. A stop mechanism 33 may be provided to halt application of the suction device 31 upon collection of a predetermined quantity of fluid in the fluid collection device 32. Interrupting the application of suction to the appliance 29 is desirable to prevent exsanguination in the unlikely event a blood vessel ruptures under the tissue cover 18 during treatment. If, for example, a blood vessel ruptures in the vicinity of the damaged bone tissue 24, a shut-off mechanism would be useful to prevent the vacuum system 30 from aspirating any significant quantity of blood from the patient. As a safety feature, various mechanical or electrical detection mechanisms may be employed to detect the level of exudate in the fluid collection device 32.

The appliance 29 includes a fluid-impermeable tissue cover 18 in the form of a flexible, adhesive, fluid impermeable polymer sheet for covering and enclosing the damaged bone tissue 24 at the damaged bone tissue site. The tissue cover 18 includes an adhesive backing 20 which functions to seal the tissue cover 18 to the attachment site 22 around the periphery of wound 24 to provide a generally gas-tight or fluid-tight enclosure over the damaged bone tissue 24. The tissue cover 18 must have sufficient adhesion to form a fluid-tight or gas-tight seal 19 around the periphery of the damaged bone tissue and to hold the tissue cover 18 in sealed contact with the attachment site 22 during the application of suction or reduced pressure.

The appliance 29 also includes a porous tissue screen 10 which is placed proximate the damaged bone tissue 24. The size and configuration of the tissue screen 10 can be adjusted to fit the treatment site. It can be formed from a variety of porous materials. The material maybe sufficiently porous to allow oxygen to reach the damaged bone tissue. The tissue screen 10 may be in the form of an open-cell polymer foam, such as a polyurethane foam, which is sufficiently porous to allow gas flow to and/or from the damaged bone tissue 24. Foams may be used that vary in thickness and rigidity, although it may be desirable to use a spongy material for the patient's comfort if the patient must lie upon the appliance during treatment. The foam may also be perforated to enhance gas flow and to reduce the weight of the appliance. As shown in FIG. 1, the screen 10 is cut to an appropriate shape and size to fit within a region about the damaged bone tissue 24. Alternatively, the screen may be sufficiently large to overlap the surrounding attachment site 22.

The appliance 29 also includes a suction port in the form of a hollow suction tube 12 that connects with the vacuum system 30 to provide suction within the sealed enclosure. The suction tubing 12 serves as a suction port for appliance 29. An end segment 12a of the tubing 12 is embedded within the foam screen 10 for providing suction or reduced pressure within the enclosure provided under the tissue cover 18. Embedding the open end of segment 12a of tubing 12 within the interior of the foam screen 10 permits the foam screen 10 to function as a shield to help prevent the tissue cover 18 from being inadvertently sucked into sealing engagement with the open end of the tube thereby plugging the tube 12 and restricting gas flow. The tube segment 12a embedded within the foam screen 10 may desirably include at least one side port 14 for positioning within the interior of the foam screen 10 to promote substantially uniform application of reduced pressure throughout the enclosure. Positioning the side port 14 of tube segment 12a within the interior of the foam screen 10 permits the foam screen 10 to function as a shield for the side port to thereby prevent the tissue cover 18 from being sucked into the side port 14 and thereby restricting gas flow. The open cells of the foam screen 10 facilitate gas flow throughout the enclosure. In addition, the foam screen 10 functions to hold the tissue cover 18 generally out of contact with the damaged bone tissue 24 during the application of suction within the enclosure.

Tubing 12 and tube segment 12a may be sufficiently flexible to permit movement of the tubing but are sufficiently rigid to resist constriction when reduced pressure is supplied to the appliance 29 or when the location of the damaged bone tissue 24 is such that the patient must sit or lie upon the tubing 12 or upon the reduced pressure appliance 29. The screen-tube assembly comprising the foam screen 10 and the tube 12 may be fabricated by snaking the end of the tube segment 12a through an internal passageway in the foam screen 10 such as by pulling the end of the tube segment 12a through the passageway using forceps. Alternatively, fabrication of the screen-tube assembly may be accomplished by suspending the end of the tube segment 12a into a suitable mold or form and then blowing foam into the mold or form to embed the tube end segment 12a within the blow-molded foam screen. The screen-tube assembly 12 and 10 is preferably prepared prior to use under sterile conditions and then stored in an aseptic package.

In order to use the reduced pressure appliance 29 at the site of the damaged bone tissue 24, the flexible, gas-impermeable, adhesive tissue cover 18 is secured in position at the damaged bone tissue site overlying the foam screen 10 disposed within the damaged bone tissue 24. The tissue cover 18 is secured and sealed to the surrounding attachment site 22 by an adhesive layer 20 on the under surface of the tissue cover 18 to form a gas-tight seal 19 around the periphery of the damaged bone tissue 24. The tissue cover 18 also provides a gas-tight seal around the tubing 12 at the feed through location 22a where the tubing 12 emerges from beneath the tissue cover 18. The tissue cover 18 is preferably formed of a fluid impermeable or gas impermeable flexible adhesive sheet such as Ioban, a product of the 3M corporation of Minneapolis, Minn.

Predetermined amounts of suction or reduced pressure are produced by the suction device 31. The suction device 31 is preferably controlled by a control device 44 such as a switch or a timer which may be set to provide cyclic on/off operation of the suction device 31 according to user-selected intervals. Alternatively, the suction device 31 may be operated continuously without the use of a cyclical timer. The control may also include a pressure selector to enable the amount of suction produced by the system to be adjusted so that a suitable sub-atmospheric pressure may be created within the chamber. Operation of the vacuum system 30 may be controlled to permit graduated increases in the amount of vacuum applied or graduated decreases in the amount of vacuum applied.

A method of treatment of damaged bone tissue in accordance with the present invention can be carried out by securing a reduced pressure appliance to the treatment site as previously shown and described, and then maintaining a substantially continuous or cyclical reduced pressure within the appliance until the damaged bone tissue has reached a desired improved condition. A selected state of improved condition may include formation of a neo-osteoid tissue. It may be preferable to change the appliance periodically, such as at 48 hour intervals, during treatment, particularly when using appliances incorporating a screen on or in the damaged bone tissue. The method is preferably practiced using a reduced pressure ranging from 0.01 to 0.99 atmospheres, and more preferably practiced using a reduced pressure ranging between 0.5 to 0.8 atmospheres. The time period for use of the method on damaged bone tissue may preferably be at least 12 hours, but can be, for example, extended for one or more days.

Supplying reduced pressure to the appliance in an intermittent or cyclic manner may also be desirable for treating damaged bone tissue. Intermittent or cyclic supply of reduced pressure to an appliance may be achieved by manual or automatic control of the vacuum system. A cycle ratio, the ratio of “on” time to “off” time, in such an intermittent reduced pressure treatment may be as low as 1:10 or as high as 10:1. For example, a useful ratio may be approximately 1:1, applied in alternating 5 minute intervals of reduced pressure supply and non-supply.

A suitable vacuum system for use in the method includes any suction pump capable of providing at least 0.1 pounds of suction to the damaged bone tissue, and preferably up to three pounds suction, and most preferably up to fourteen (14) pounds suction. The pump can be any ordinary suction pump suitable for medical purposes that is capable of providing the necessary suction. The dimension of the tubing interconnecting the pump and the reduced pressure appliance is controlled by the pump's ability to provide the suction level needed for operation. A ¼ inch diameter tube may be suitable.

### EXAMPLE

A 61 year old male fell from a ladder while loading steel at his place of employment, sustaining an open pilon variety fracture on the right distal tibia and fibula and a nondisplaced tibial plateau fracture on the contralateral side. According to the referring surgeon, he was initially managed with a splint for his tibial plateau fracture and a surgical debridement of his open distal tibial (pilon) fracture with open reduction and internal fixation of the fractured fibula, reduction and percutaneous screw fixation of the portion of the fracture extending to the joint surface, application of dressings to the open fracture wound, and application of a long leg splint. This was followed four days later by a re-inspection and irrigation of the wound, cancellous bone grafting of the defect, and application of an external fixator (non-bridging hybrid variety with thin, tensioned wires distally.)

The patient was transferred to a rehabilitation facility, and orthopedics was consulted to evaluate his leg injury. At this time, the wound appeared red and was draining. A debridement and irrigation of the wound, including removal of the infected graft from the defect in the tibia, was undertaken with application of antibiotic beads as well as application of a vacuum assisted closure system as shown in FIG. 1. The vacuum system was placed at the wound site to form an enclosure about the tibial defect. The foam section was placed proximate the tibial defect but out of contact with the tibial defect. The foam section, however, could be placed in contact with the tibial defect if so desired. The vacuum was set to −125 mmHg pressure, that is 125 mmHg below atmospheric pressure. The patient utilized the system for approximately three and a half months, with the bulk of this time in the home setting. The foam section was changed at 48-hour intervals. The patient was placed on a course of culture directed intravenous antibiotics for six weeks while undergoing the vacuum treatment. The antibiotic beads were removed at one month following the initial debridement. During this time, he was evaluated for a free flap with the aim toward coverage of his wound whose base consisted of the defect in the tibia.

The patient had a long history of cigarette smoking (50 pack years), with chronic obstructive pulmonary disease and atherosclerotic distal leg vessels which made him a poor candidate for the procedure. At this point, the option of below knee amputation with early prosthetic fitting was recommended with the aim toward early functional restoration, but the patient refused this option. It was because of the contraindications to free flap coverage and the patient's refusal to undergo an amputation that the relatively prolonged vacuum treatment was elected and continued on an outpatient basis. Vacuum treatment was continued on an outpatient basis under the conditions mentioned above, with weekly follow-up visits to the patient's orthopaedic surgeon. Over the span of the subsequent six weeks, the metadiaphyseal defect of the tibia filled in with what appeared on the surface to be healthy granulation tissue which spontaneously epithelialized. One would anticipate a persisting nonunion in these circumstances. For the defect to have spontaneously healed with bone was not anticipated. The spontaneous formation of specialized tissue at the surface (epithelium) mirrored the spontaneous formation of specialized tissue at the level of the tibial defect (bone). The progenitor of bone in the defect was a neo-osteoid tissue whose formation was encouraged by the vacuum treatment.

The terms and expressions which have been employed are used as terms of description and not of limitation and there is no intention in the use of such terms and expressions of excluding any equivalents of the features shown and described, or portions thereof, but it is recognized that various modifications are possible within the scope of the claimed invention.

What is claimed is:

1. A method for administering a reduced pressure treatment to a damaged bone tissue, comprising the steps of:
(a) providing an impermeable cover adapted to cover and enclose the damaged bone tissue and adapted to maintain reduced pressure at the site of the damaged bone tissue;
(b) providing a seal adapted to seal said cover to tissue surrounding the damaged bone tissue;
(c) providing reduced pressure supply means for connection to a source of suction, said reduced pressure supply means cooperating with said cover to supply said reduced pressure beneath said cover;
(d) providing a screen adapted to prevent contact between the cover and the damaged bone tissue, said screen being located between said damaged bone tissue and said cover;
(e) applying a reduced pressure under the cover to the damaged bone tissue; and
(f) maintaining the reduced pressure until new bone tissue has grown at the damaged bone tissue to provide a selected stage of healing.

2. A method for applying reduced pressure to a damaged bone tissue beneath a fluid-impermeable seal comprising the steps of:
providing an open cell polymer foam section positioned beneath said seal to overlie the damaged bone tissue such that said reduced pressure is maintained within said foam and applied to the damaged bone tissue;
providing a flexible tube having an inlet end inserted into said open cell polymer foam section and an outlet end extending from beneath said seal for supplying said reduced pressure; and
maintaining the applied reduced pressure to the damaged bone tissue until the new bone tissue has grown at the damaged bone tissue to provide a selected stage of healing.

3. A method for facilitating the healing damaged bone tissue comprising the steps of:
providing a vacuum means for creating a reduced pressure on a treatment area including and surrounding the damaged bone tissue;
providing a sealing means operatively associated with said vacuum means for maintaining said reduced pressure on said damaged bone tissue by contacting the skin surrounding said damaged bone tissue, wherein said sealing means comprises a fluid-impermeable cover;
providing a screen means for positioning at the damaged bone tissue within the sealing means for delivering the reduced pressure to the damaged bone tissue; and
applying a reduced pressure to the damaged bone tissue through the screen means; and
maintaining the reduced pressure until new bone tissue has grown at the damaged bone tissue to provide a selected stage of healing.

4. A method of healing a bone defect comprising the steps of:
(a) applying a reduced pressure to a bone defect; and
(b) maintaining the reduced pressure until new bone tissue has grown at the defect to provide a selected stage of healing.

5. The method according to claim 4 comprising the step of applying an antibiotic to the bone defect.

6. The method according to claim 5 wherein the step of applying an antibiotic comprises the step of applying antibiotic beads.

7. The method according to claim 4 wherein the step of applying reduced pressure comprises the steps of applying a cover over the bone defect and supplying reduced pressure beneath the cover for application of the reduced pressure to the bone defect.

8. The method according to claim 7 wherein the step of applying a cover comprises the step of sealing the cover about a periphery of the bone defect.

9. The method according to claim 7 comprising the step of supplying a screen beneath the cover.

10. The method according to claim 7 comprising the step of supplying a foam screen beneath the cover.

11. The method according to claim 10 wherein the step of supplying a foam screen comprises supplying an open cell foam beneath the cover and wherein the step of applying reduced pressure includes applying reduced pressure internally of the foam screen.

12. A method of treating a bone defect comprising the steps of:
(a) applying a reduced pressure to the bone defect, wherein said applying step comprises the steps of:
(i) locating an impermeable cover over the bone defect, said cover having a suction port;
(ii) sealing the periphery of said impermeable cover to tissue surrounding the bone defect; and
(iii) operably connecting said suction port with a vacuum system for producing said reduced pressure; and
(b) maintaining said reduced pressure until the bone defect has progressed toward a selected stage of healing, the selected stage of healing including formation of neo-osteoid tissue.

13. A method of treating a bone defect comprising the steps of:
applying a reduced pressure to the bone defect; and
maintaining said reduced pressure until the bone defect has progressed toward a selected stage of healing, the selected stage of healing including formation of neo-osteoid tissue.
